(19) United States
(12) Patent Application Publication
(10) Pub. No.: US 2021/0174273 A1
(43) Pub. Date: Jun. 10, 2021

(54) RAPID PROTOTYPING MODEL

(71) Applicant: Battelle Memorial Institute, Columbus, OH (US)

(72) Inventors: Jarrod R. Olson, Columbus, OH (US); Tyler Coy, Columbus, OH (US); Po-Hsu Chen, Columbus, OH (US); Adrienne Cocci, Columbus, OH (US); Amir Rahimi, Columbus, OH (US); J. Elizabeth Jackson, Columbus, OH (US); Amanda Morgan, Columbus, OH (US); Jeffrey J. Geppert, Columbus, OH (US); Nancy J. McMillan, Columbus, OH (US)

(21) Appl. No.: 16/952,162

(22) Filed: Nov. 19, 2020

Related U.S. Application Data
(60) Provisional application No. 62/945,633, filed on Dec. 9, 2019.

(51) Int. Cl. G06Q 10/06; G06Q 50/22

(52) U.S. Cl. CPC G06Q 10/067; G06Q 50/22

(57) ABSTRACT

Rapid prototyping of a process in a business domain, such as a healthcare domain or the United States healthcare domain, is disclosed. A computer is programmed to perform discrete event simulation (DES) of a scenario in the business domain. The DES is defined by a plurality of resources having resource attributes, a plurality of events having event attributes, and a plurality of entities having entity attributes. The computer is programmed to implement a DES simulator configured to run the scenario including performing time steps per resource in which the entities experience events.

This application claims the benefit of U.S. Provisional Application No. 62/945,633 filed Dec. 9, 2019 and titled “RAPID PROTOTYPING MODEL”. U.S. Provisional Application No. 62/945,633 filed Dec. 9, 2019 and titled “RAPID PROTOTYPING MODEL” is incorporated herein by reference in its entirety.

### BACKGROUND

The following relates to the discrete event simulator arts, healthcare policy development arts, healthcare cost management arts, and related arts.

### BRIEF SUMMARY

In one illustrative embodiment, a rapid prototyping system is disclosed for prototyping a process in a business domain. The rapid prototyping model comprises a computer programmed to perform discrete event simulation (DES) of a scenario in the business domain, the DES being defined by a plurality of resources having resource attributes, a plurality of events having event attributes, and a plurality of entities having entity attributes, the computer programmed to implement a DES simulator configured to run the scenario including performing time steps per resource in which the entities experience events. In some embodiments, the DES simulator is implemented in AnyLogic or Python. The computer may be further programmed to generate a synthetic population of entities upon which the DES simulator runs the scenario, and may be further programmed to generate outcomes for the scenario based on the running of the scenario, which may for example be presented on a display (e.g., an LCD display, plasma display, OLED display, or so forth). The outcomes for the scenario may include aggregated outcomes for the population, and/or disaggregated outcomes for defined segments of the population. The computer in some embodiments is programmed to run a baseline scenario that does not include a proposed innovation to the business domain, and an innovation scenario that includes the proposed innovation to the business domain, and the generated outcomes then include at least one comparison of the outcomes for the innovation scenario versus the outcomes for the baseline scenario. The computer may be further programmed to perform uncertainty analysis on the generated outcomes for the scenario, such as a scenario-based uncertainty analysis in which predefined scenarios are run by the DES simulator to test limits of the innovation, or a Monte Carlo-based uncertainty analysis in which scenarios generated by Monte Carlo sampling are run by the DES simulator to generate distributions of the outcomes. In some illustrative embodiments, the business domain is a healthcare domain, or more specifically the United States healthcare domain.

In another illustrative embodiment, a rapid prototyping method is disclosed for prototyping a process in a business domain. The rapid prototyping method comprises: using a computer, running a discrete event simulation (DES) of a scenario in the business domain, the DES being defined by a plurality of resources having resource attributes, a plurality of events having event attributes, and a plurality of entities having entity attributes, the running of the DES of the scenario including performing time steps per resource in which the entities experience events; and generating outcomes for the scenario based on the DES of the scenario. In some embodiments, the DES is implemented in AnyLogic or Python. In some embodiments, the method further includes, using the computer, generating a synthetic population of entities upon which the computer runs the DES of the scenario. In some embodiments, the computer is programmed to run DES of (i) a baseline scenario that does not include a proposed innovation to the business domain, and (ii) an innovation scenario that includes the proposed innovation to the business domain, and the generated outcomes include at least one comparison of the outcomes for the innovation scenario versus the outcomes for the baseline scenario. Some embodiments further include, using the computer, performing uncertainty analysis on the generated outcomes for the scenario, such as scenario-based uncertainty analysis in which the computer runs DES of predefined scenarios to test limits of the innovation or Monte Carlo-based uncertainty analysis in which the computer runs DES of scenarios generated by Monte Carlo sampling to generate distributions of the outcomes. In some illustrative embodiments, the business domain is a healthcare domain, or more specifically the United States healthcare domain.

In another illustrative embodiment, a non-transitory storage medium stores instructions readable and executable by a computer to perform a rapid prototyping method as set forth in the immediately preceding paragraph.

### BRIEF DESCRIPTION OF THE DRAWINGS

FIG. 1 presents an illustrative decision-making process, per Center for Medicare and Medicaid Innovation (CMMI) Payment and Service Delivery Innovations, from review of policies and procedures.

FIG. 2 diagrammatically shows a rapid prototyping system for prototyping a process in a business domain.

FIGS. 3A, 3B, and 3C present an example diagram showing elements of key simulator definitions and associated classes/attributes.

### DETAILED DESCRIPTION

Rapid Prototyping Models disclosed herein comprise simulators (e.g. Python-based) of a healthcare policy system that models individuals moving through the healthcare system as a series of discrete events. Each event has a set of probabilistic outcomes and each simulated person has a set of attributes modifying that probability distribution. For economically driven decisions, the decision-making agent utilizes a net present value approach for their expected costs and benefits to make a cost-benefit analysis within a given budget constraint. As healthcare services are utilized, they are recorded using actual Centers for Medicare & Medicaid Services (CMS) claim codes (or other claim codes, e.g. insurance payer claim codes, which may be specific to a particular insurer, or so forth) and other data is recorded in high fidelity to allow detailed drill-down into the cause and effect behavior within the system. This allows for analysis of policy changes (specified as a scenario) while incorporating realistic outcome likelihoods with the potential for emergent behavior. All discrete event specifications are drawn from peer-reviewed literature, with limited exceptions that utilize transparent and clearly documented assumptions.

Healthcare policy changes, particularly in the arena of alternative payment models, are difficult to assess before pilot testing and current approaches depend heavily on highly biased expert judgement. CMS has spent billions of dollars designing, testing, and evaluating alternative payment models since 2012, but has shown a demonstrable effect (positive or negative) in a small set of their 70+ pilots, which take 3-5 years, expose patients, providers, and the government to risk, and cost a lot. The disclosed tool successfully replicated one of these studies and would allow for rapid testing of alternative designs to find the version most likely to succeed. This will also pay dividends during evaluation of models to help understand the internal workings of pilots when data is not available, and to drive new data collection during evaluations. By reducing the likelihood of testing an alternative payment model that is likely to fail, and improving the data collection during pilot implementation, this will reduce the failure rate, and increase the likelihood of identifying a positive or negative effect from the pilot.

The illustrative discrete event simulation disclosed herein is applied in the U.S. healthcare space, and Discrete Event Simulation with economic decision-making and healthcare service delivery is incorporated. Illustrative embodiments of the Discrete Event Simulation are focused on alternative payment and service delivery models.

In a workflow of operations where the simulation is started during the alternative payment model design process, the parameters of the proposed innovation are provided early to the development team and simulations on alternative designs start immediately. A combination of expert-provided scenarios and Monte Carlo simulations identify differences in proposed designs and the design reports are provided to the designers with recommendations about the relative likelihood of success for each design concept. During evaluation, as data from the real-world is provided, increasingly precise simulation runs will help to identify gaps in data collection and drive additional analysis into the real-world results, with a final product where the simulation has been calibrated to real-world implementation data.

Disclosed herein are embodiments of a Healthcare Technology Innovation Laboratory (HTIL) Rapid-Prototyping Model (RPM) for Payment and Service Delivery Models. Innovations in healthcare policy (e.g. payment and service delivery models) are currently pilot tested with small-scale comparative evaluations that are costly to implement, take several years to complete, expose participants to risk of harm, lack statistical power, and often result in inconclusive findings regarding the impact of the pilot. RPM will be a high-fidelity simulation capability that allows rapid prototyping of policy decisions for our clients before putting human subjects at risk. It will reduce cost by focusing on high value activities, and risk of failure for effectiveness studies by reducing uncertainty in the selection of innovations to pilot. The overall guiding approach is to build a simulation framework that is object-oriented, enabling analysts to add new, detailed models to test a variety of specific policy innovations in a short cycle to inform decisionmakers of the expected impacts of design decisions. The three main use cases are evaluation (i.e. evaluating the expected impact of an intervention to support decision-making about approving pilot testing), evaluation support (i.e. informing evaluation design by identifying where in the body of evidence there are the most significant gaps), and program integrity (i.e. using probabilistic information to identify situations where a performer is greatly exceeding expectations).

The RPM Framework is a design specification that enables integration and re-use of detailed event models and supporting infrastructure to model outcomes across a variety of policy domains, particularly payment and service delivery innovations. By re-using the generalizable elements of the framework, like iteration logic, synthetic populations, common healthcare events, and standards for interconnecting specific event models, the cost and time involved in the initial RPM Framework development can be leveraged for future problems. This will reduce the time from start to finish of simulating and analyzing the policy innovation. Documentation of the process will allow for full replications by teams outside of the analysis team and interoperability will be maximized by storing outputs in accessible, well-documented data storage formats.

The framework relies primarily on discrete event simulation, where a problem is abstracted as a process of events where a set of actors conditions the outcome of the event. An actor may be a patient, a doctor, or even an organization. Each “discrete event” comprises a series of options drawn from an independent probability distribution. Accordingly, at each event, an actor's features modify the probability distribution and a random draw is utilized to estimate their outcome. Actor features for individuals may include race/ethnicity, insurance status, gender, etc., while features for an organization might include region, volume of care, specific capabilities, etc. While each event is independent (making parameterization easier), the full path through an iteration of the model (some period) contains dependencies because different paths include and exclude encountering some sets of events for a given actor.

A synthetic population of actors (i.e. entities or objects) is estimated based on known characteristics (drawn from surveys, census, and other sources of data) and then each individual in the synthetic population works through the model in defined timesteps. Because the probability distribution of each function is conditional upon the actors' features, stratification is possible at the most micro-level of detail defined for an actor in the synthetic population. This population itself is generalizable a part of the framework and can be re-used with appropriate modifications (if there are case-specific risk factors to incorporate, for example).

As outlined in Table 1, three types of typical use cases are: Evaluation; Evaluation Preparation; and Program Integrity. In the Evaluation use case, the HTIL RPM is used to evaluate the expected impact of a given intervention. In the Evaluation Preparation use case, the HTIL RPM is used to design data collection strategies targeting the areas of greatest uncertainty or sensitivity to implementation for an intervention by assessing the evidence base quantitatively to support post-pilot evaluation of actual impact. In the Program Integrity use case, the HTIL RPM is used to assess the likelihood of achieving a given real-life scenario and then working back to re-construct the steps leading to that outcome to validate claims and other data.

TABLE 1
General use cases describing HTIL RPM expected utilization

| ID | Name | Description |
|---|---|---|
| 1 | Evaluation | HTIL RPM is used to evaluate the expected impact of a |
| | | given intervention |
| 2 | Evaluation | HTIL RPM is used to design data collection strategies |
| | Prep | targeting the areas of greatest uncertainty or sensitivity to |
| | | implementation for an intervention by assessing the |
| | | evidence base quantitatively to support post-pilot |
| | | evaluation of actual impact |
| 3 | Program | HTIL RPM is used to assess the likelihood of achieving a |
| | Integrity | given real-life scenario and then working back to re- |
| | | construct the steps leading to that outcome to validate |
| | | claims and other data |

The DES is performed, in some illustrative applications, in the context of a development process that (for example) follows the Agile Scrum methodology, where key user stories are defined as performance requirements and developers work in parallel to build components of the model during time-bound development sprints (typically two weeks). Each sprint focuses on producing a “minimally viable product” that represents a fully functional prototype. All code and relevant datasets will be maintained in a version-controlled repository. JIRA (a proprietary issue tracking product developed by Atlassian that allows bug tracking and agile project management) is suitably utilized for tracking user stories, tasks, and sprints. This is merely an illustrative implementation.

The purpose of sprints, following the development of initial infrastructure development, is to reduce forecasting uncertainty or increase performance. Forecasting uncertainty is the quantitative assessment of unknown information and impacts the precision of estimates (forecasting uncertainty is typically envisioned as confidence intervals). Each development sprint should add detail to existing “black boxes” where large assumptions are made and process steps are lumped together for simplicity. The improvement in forecasting uncertainty may apply to the aggregated outcomes (i.e. full population level) or the disaggregated outcomes (i.e. for specific stratified populations, i.e. defined segments of the population). Performance is defined as the amount of time required to prepare data inputs, run the simulator, and export the results.

The Modeling Domain is further described in the following, for a nonlimiting illustrative context of the U.S. healthcare system, in which reimbursement claims are described using Centers for Medicare & Medicaid Services (CMS) claim codes. More generally, the disclosed RPM can be employed in other healthcare contexts (e.g., outside the U.S.), and/or employing other types of claim codes and/or other healthcare reimbursement frameworks and/or descriptors. Even more generally, the disclosed DES simulators may find application in a wide range of other business domains, such as transportation policy development, transportation system cost management, educational policy development, educational system cost management, and so forth. The following is to be understood to be a nonlimiting illustrative example.

The core modeling domain for the RPM Framework is healthcare payment and service delivery model innovations, and, for this nonlimiting illustrative example, assumes a context of U.S. healthcare in which reimbursement employs CMS claim codes. A brief history is provided of Medicare and Medicaid reimbursement models and their connection to service delivery, and the new role the Center for Medicare and Medicaid Innovation (CMMI) plays in developing innovations to address shortcomings in this reimbursement system. Some of the general characteristics of the payment and service delivery model innovations that CMMI has put forward are also described. Decision-making criteria that CMMI faces to start implementation of a new pilot or to continue the pilot are also described.

Since Medicare and Medicaid were established in the 1960s, payment has relied on the concept of a Fee-For-Service payment model, where reimbursement is based on inputs to the process rather than the most appropriate care over an episode of illness or over the course of a year. Fee-for-service incentivizes providers to provide a large volume of services, even when better, lower-cost methods exist to treat a condition. In 1988 Medicare was authorized by Congress to test a new reimbursement model, the Coronary Artery Bypass Graft (CABG) bundled payment. Another model was tested in 2005 looking at Physician Group Practice payment models. Both reforms tied reimbursement to performance against quality measures. In the 2009 Affordable Care Act, Section 3021 created the Center for Medicare and Medicaid Innovation (CMMI) and gave it broad authorization to test new models that tie pay to performance against quality measures or implement changes to service delivery models. CMMI is authorized to test models brought forward by legislation (i.e. from legislators) or other sources based on the judgment of the Secretary of Health and Human Services (HHS).

Since the passage of the Affordable Care Act, CMMI has planned pilots for more than 70 new payment and service delivery models and completed 12 (as of February 2017, by our count of status=“No Longer Active” from the CMMI website, excluding 2 entries that are just summaries of categories of pilot groups). These model innovations target a variety of possible innovations that must meet certain conditions. At its core, a model innovation must either (1) reduce costs without affecting quality; (2) improve quality without affecting costs; or (3) improve quality and reduce costs. A variety of other decision criteria are utilized for prioritizing these model innovations, including the types of people served, the overall scale of the innovation's impact, and other factors outlined in CMMI's authorizing legislation.

The model innovations utilize interventions that fall broadly into the categories Evaluation; Evaluation Preparation; and Program Integrity, as previously described (note, this is drawn from a sample of completed models and models that have not yet been implemented accounting for 30 of the 70+ models), with slight variations. The definitions and categories were assigned by analysts based on text descriptions available from the pilot website or corresponding Fact Sheet to categorize similar interventions. These interventions are often combined to incentivize certain behaviors.

Interventions target a range of groups, typically Hospitals (10), Providers (6), Physicians (5), Accountable Care Organizations (4), and Beneficiaries (4). When an intervention targets a more specific group, it is coded accordingly (for example, a physician is also a provider, so if no more specific target information is provided, then that physician will be counted as a provider, not a physician). The groups that benefit directly from these interventions (other than financially) are primarily Medicare beneficiaries (only 5 address Medicaid or CHIPS) that range from broad populations to more discrete sub-groups. One of the decision factors affecting approval of a pilot is its impact disadvantaged groups. Some interventions specifically target only a subset of health conditions.

Outputs (see Table 2) are generated by an intervention and contribute to an outcome. Detailed outputs are not available from the described overview analysis of the payment and service delivery models and in many cases had to be inferred from general pilot descriptions. In this discussion, we focus on outputs specified by the summary descriptions. Outputs will be produced at a varying rate depending on the intervention and level of effort (i.e. how much a participating organization contributes). These outputs theoretically will drive changes for outcomes, defined in Tables 3 and 4.

TABLE 2
Innovation types, descriptions, and
frequency from sample of CMMI models

| | # of | |
|---|---|---|
| | Innovations | |
| Innovation | (not mutually | |
| Category | exclusive) | Notes |
| Advance | 5 | Interventions that provide advance |
| Payment | | payment for a block of services (also |
| | | called capitation), or for investment in |
| | | infrastructure or for investment in |
| | | infrastructure to support quality |
| | | improvement activities |
| Bundled | 6 | Reimbursement is based on an |
| Payment | | episode of care, typically involving an |
| | | initial admission and ending with |
| | | rehabilitation activities (e.g., knee |
| | | surgery) |
| Commu- | 1 | Interventions targeted broadly around |
| nication | | communications |
| Data Sharing | 5 | Interventions that explicitly include data |
| | | sharing between parties that normally |
| | | would not share data. Often this is cost |
| | | and quality data. |
| Directed | 1 | Interventions that specifically re-direct |
| Payments | | traditional fee-for-service payments to |
| | | different parties |
| Learning and | 5 | Interventions that include an aspect of |
| Diffusion | | education through a centralized |
| Program | | learning and diffusion program |
| Patient | 3 | Education targeting the patients as an |
| Education | | intervention |
| Patient | 1 | Interventions that aim to engage |
| Engagement | | patients in communication and shared |
| | | decision-making, as opposed to |
| | | education |
| Pay for | 16 | Pay is specifically tied to performance |
| Performance | | against a set of quality indicators |
| Population- | 3 | Payment is based on a global budget |
| Based | | for a population based on its risk |
| Payment | | factors, a slight variation on Advance |
| | | Payment |
| Practice | 7 | Specific approaches to treatment are |
| Change | | taught or incentivized directly to |
| | | change cost or quality factors |
| Regulatory | 2 | Revisions in regulation necessary to |
| Revision | | allow or incentivize new behaviors |
| | | (typically called a waiver) |
| Shared Savings | 8 | Interventions that share cost savings |
| | | due to reimbursement savings between |
| | | CMS and another entity, typically a |
| | | doctor, hospital, or patient |
| Technical | 3 | Training is provided directly to |
| Assistance | | participating organizations, staff, etc. |

TABLE 3
Outcome types, frequency, and definitions
for sampled CMMI models

| | #Outputs | |
|---|---|---|
| Category | Mapped | Definition |
| Money | 15 | Financial changes |
| Education | 13 | Learning or educational opportunities such as |
| | | trainings |
| Commu- | 8 | Outreach, etc. that is not labeled as education |
| nication | | |
| Service | 7 | Changes in the types or numbers of medical |
| | | services provided |
| Collab- | 5 | Changes in collaborative activities between |
| oration | | disparate groups |
| Data | 5 | Changes in data access or provision |
| Policy | 5 | Changes in policy such as new statements of |
| | | support for a given approach |
| Risk | 4 | Changes in risk burden (i.e., who takes on |
| | | the risk for poor performance) |
| Legal | 4 | Changes in legal obligations, such as |
| | | contracts |
| Measurement | 4 | Changes in measurement approaches |
| Resources | 3 | Changes in availability of resources, |
| | | including human capital |
| Planning | 2 | Changes in planning approach |
| ?? | 2 | Undefined |
| Organization | 1 | Changes in organizational structure |

TABLE 4
Outcomes types, frequencies, and definitions
from sampled CMMI models

| | # Outcomes | |
|---|---|---|
| Category | Mapped | Definition |
| Quality | 41 | Outcomes related to care quality or |
| | | patient outcomes |
| Cost | 20 | Outcomes related to financial |
| | | outcomes |
| Care Coordination | 14 | Outcomes related to coordinating |
| | | care across different caregivers |
| Patient Knowledge | 6 | Outcomes related to informing |
| | | patients or otherwise educating them |
| Efficiency | 6 | Outcomes focused on reducing |
| | | redundancy or linking cost and |
| | | quality explicitly |
| Practice | 5 | Outcomes related to long-term |
| | | change in practice |
| Education | 2 | Outcomes related to education for |
| | | providers |
| Patient Outcome | 2 | Outcomes for patient specific |
| | | achievement (e.g., weight loss, |
| | | cardiac fitness) |
| Infrastructure | 1 | Outcomes for changes in |
| | | infrastructure |
| ?? | 1 | Undetermined how to categorize: |
| | | “Decreased incentive for |
| | | hospitalization” |
| Coverage | 1 | Outcomes related to changes in |
| | | coverage. |

Outcomes are the terminal goals of a given intervention. The outcomes may be presented on a display (e.g., an LCD display, plasma display, OLED display, or so forth), printed by a printer or other marking engine, stored in a file, and/or otherwise utilized, They are the things that an intervention aims to change, but cannot do directly. They depend on the outputs and a variety of other factors such as implementation fidelity and other intervening variables. For this modeling domain, the primary outcomes of interest are quality and cost.

In addition to understanding the types of activities and outcomes at a general level, it is helpful to understand the decision-making environment that works to approve these pilots. The key questions CMMI tends to grapple with (again, in this specific example directed to DES simulation of CMS) in decision-making are outlined as follows. First order issues (i.e. must do at all) include reducing costs as defined in CN: USG, 2012, 42 U.S.C. 1315a Section 1115a, or increasing quality of care, or both. These issues can be evaluated, and fit into the listing of allowed model types from CN: USG, 2012, 42 U.S.C. 1315a Section 1115a. Second order issues (i.e. it should do as much of this as possible, in no particular order) include giving providers flexibility to exercise judgment, furthering the aims of health equity (i.e. increasing care to the disadvantaged and not excluding anyone new), utilizing health information technology (IT), contributing to integration and care coordination among teams, enhancing patient decision-making and agency, and communicating more effectively with patients. As the pilot is underway, a contractor is responsible for implementing the innovation and a separate, independent contractor is responsible for evaluating the pilot. These evaluations rely heavily on quality measures representing outcomes and treat the model innovation as a black box without detailed process modeling (for the most part). The results of the pilot evaluation inform the decision to shut down, expand, or continue in its current state a given pilot.

To constrain the possible set of issues that the HTIL RPM will address, we have identified a set of key questions that it will answer. In each case, the question is framed as “Given the set of assumptions and modeling constraints selected,”. The list of questions includes the following.

What was the change in specified quality measures for the pilot groups, compared to the baseline/comparison group (fee for service)? In addressing this question, the HTIL RPM should provide the ability to stratify down to core groups (race, SES, gender, Medicare, Medicaid, rural, urban, etc.) depending on the focus of the pilot.

What was the change in cost for the pilot groups, compared to the baseline/comparison group (fee for service)? In addressing this question, the HTIL RPM should provide the ability to stratify down to core groups (Medicare, Medicaid, provider, hospital, provider group, patient, community of patients).

What specific aspects of variation in implementation of the model (both planned and unplanned) affect outputs and outcomes and how? That is, if the model works, why does it work? For evaluation design purposes, reframe as “what are the areas of greatest uncertainty to target data collection for?” For program integrity purposes, reframe as “What is the likelihood of accomplishing a given level of improvement?”

Under what set of scenarios (or in a robust uncertainty analysis) do these results change and how can the design be optimized to maximize the values for each question?

How was patient agency and decision-making affected?

How was care coordination between specified groups affected?

These key questions will drive development of the RPM framework.

Next, the overall workflow for the RPM Framework is described, as well as its envisioned fit into the decision-making framework for CMS.

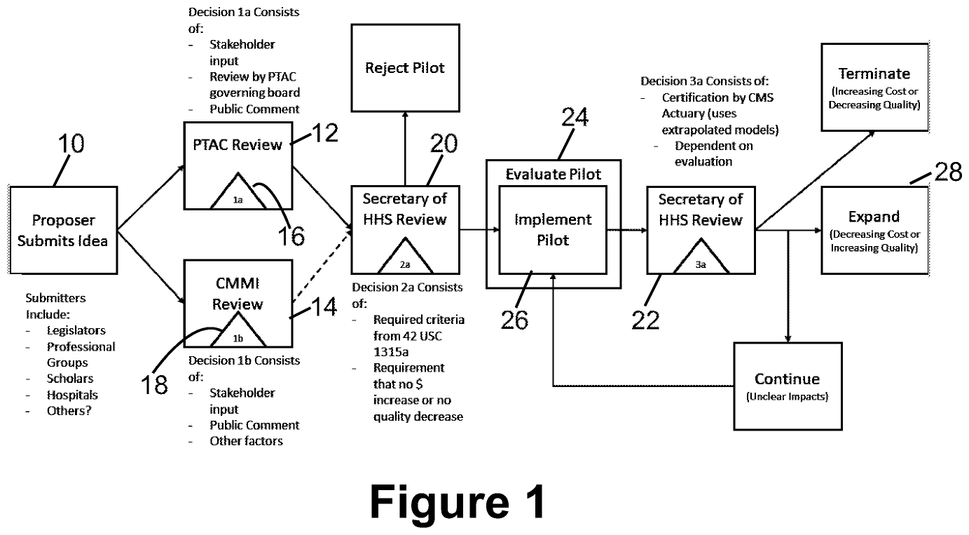

The current decision-making framework for CMS relies heavily on a stakeholder-driven process and public comment periods. FIG. 1 shows the general steps in the life-cycle of a model innovation pilot. A pilot is proposed 10 and it goes to one of two reviewing bodies 12, 14. One, PTAC (Physician-Focused Payment Model Technical Advisory Committee) 12 reviews only applications that are for Physician-Focused Payment Models. All other reviews work through another, less defined mechanism 14, typically including staff and/or contractors for CMMI. In each case, stakeholder input and public comment are key decision inputs for decision 1a 16 of the PTAC review 12 and decision 1b 18 of the CMMI review. The PTAC review 12 also relies on a governing board review with a public meeting component where they review data submitted with a given proposal. A high-level overview of the types of questions considered during an application from CMMI suggests a similar process might be suitable.

Two different gate reviewing bodies 20, 22 filter opportunities to prepare for review 20 by the Secretary of HHS. This would support Use Case 1, Evaluation 24. At this point, it is not clear if there is a defined process that involves contracting analysis out, but we assume that the opportunity exists. As a contributor to the evidence-base of a proposed idea, the RPM Framework could be leveraged as evidence by either a proposer or the review panel. Hence, HTIL RPM can be used for Use Case 2—Evaluation Preparation. Finally, as it applies to pilots 26, Use Case 3—Program Integrity would likely apply to fully implemented (i.e. “expand”) innovations 28.

The disclosed process for employing the RPM Framework involves a series of steps to parameterize existing, reusable model components and build new model components. This concept of operations is written with terminology consistent with Use Case 1, but generally applies across all three use cases. Points of departure are explicitly noted. The general proposed process is as follows.

First, background research is performed. This entails working with proposer documentation to develop a logic model describing the intervention, its expected outputs, and the expected outcomes. Key aspects of the logic model will include linkages showing how resources are converted at the intervention into outputs and how those outputs convert into outcomes. An evidence basis for these causal linkages will support the parameterization of model components.

Next, parameterization of the baseline model is performed. The baseline model represents the expected outcomes under current standards (e.g. Fee-for-service) and provides a comparison baseline. The payment and service delivery models for the baseline model are all generalizable for future model runs, but may require expansion to meet the specific needs of a given model innovation. For example, if the model has only been run against a generic set of cardiac conditions, and a comparison against the Coronary Artery Bypass Graft (CABG) intervention is proposed, the black-box elements of the baseline models for cardiac conditions may require additional detail.

Next, parameterization of the innovation model is performed. This entails, using the logic model and evidence basis, determining the core generalized components of the RPM Framework to re-use, and any previously utilized sub-models that can be re-purposed. Relevant parameters and conditions are identified for those models and sub-models to tailor to this specific policy innovation. Utilizing the evidence basis and logic model, any new sub-models are identified and developed needed.

The uncertainty analysis approach is next determined. An uncertainty analysis facilitates understanding the potential impacts of changes in parameterization of the model. Two approaches are suitably considered. In a scenario-based analysis approach, the analyst defines a-priori cases to test the limits of the model based on their understanding of model function, the logic model, and data constraints. Each of these cases (or scenarios) will be run and compared against the primary performance case. This is true of both the Baseline Model and the Innovation Model case. Alternatively, in a Monto-Carlo-based analysis approach, the range of potential uncertainties in the model are identified and Monte-Carlo methods are used to simulate them at random to estimate the distribution of expectations. This approach is most desirable as it depends less on analyst judgment.

Model runs are next performed. Based on the uncertainty analysis approach, the model is run as needed and model outputs are generated for analysis.

The results are then analyzed. Results are suitably compared between the Baseline Model and the Innovation Model, and results from the uncertainty characterization are preferably considered to make robust analysis. If needed, new uncertainty scenarios may be developed to address any difficult to explain variation.

Finally, the results are presented. In one suitable approach, a briefing document summarizing the method, the scenarios, and the model results is prepared at a level sufficient for peer-reviewed publication. Additionally or alternatively, a policy-level briefing may be prepared to communicate the results to decisionmakers in a short format appropriate for the venue and submit.

Some further illustrative examples of the HTIL RPM are next described.

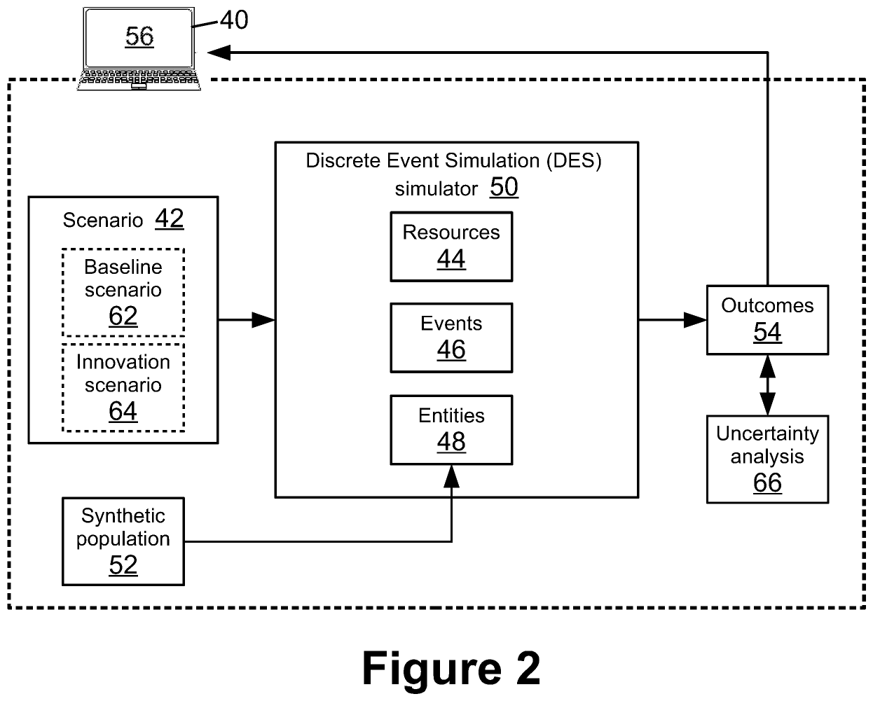

With reference to FIG. 2, the HTIL RPM utilizes Discrete Event Simulation (DES) as a primary modeling framework. The rapid prototyping system for prototyping a process in a business domain comprises a computer 40 programmed to perform discrete event simulation (DES) of a scenario 42 in the business domain. The DES is defined by a plurality of resources 44 having resource attributes, a plurality of events 46 having event attributes, and a plurality of entities 48 having entity attributes. The computer 40 is programmed to implement a DES simulator 50 configured to run the scenario 42 including performing time steps per resource 44 in which the entities 48 experience events. The computer 40 is preferably further programmed to generate a synthetic population 52 of the entities upon which the DES simulator 50 runs the scenario 42, and to generate outcomes 54 for the scenario 42 based on the running of the scenario 42. The outcomes 54 may be presented on a display 56 (e.g., an LCD display, plasma display, OLED display, or so forth), and/or printed by a printer or other marking engine, stored in a file, and/or otherwise utilized.

In some embodiments, the computer 40 is programmed to run a baseline scenario 62 that does not include a proposed innovation to the business domain, and an innovation scenario 64 that includes the proposed innovation to the business domain. The generated outcomes 54 then include at least one comparison of the outcomes for the innovation scenario 64 versus the outcomes for the baseline scenario 62. The computer 40 may then be further programmed to perform uncertainty analysis 66 on the generated outcomes for the scenario. For example, the uncertainty analysis 66 may comprise a scenario-based uncertainty analysis in which predefined scenarios are run by the DES simulator 50 to test limits of the innovation. In another approach, uncertainty analysis 66 may comprise a Monte Carlo-based uncertainty analysis in which scenarios generated by Monte Carlo sampling are run by the DES simulator 50 to generate distributions of the outcomes 54.

DES is best used when the focus of a problem is modeling a system in detail and attaining results at a micro-level (e.g. individuals with specific features/characteristics). Dynamics in DES are driven by discrete event occurrence. “Event occurrence” is defined by a specified probability distribution conditioned on the features of a given “person” in the synthetic population. Experiments are conducted by modifying process structure such as the pathways between events or the probabilities associated with a specific event outcome.

Factors driving this decision include the types of questions specified in Core Assumptions>Key Questions, the desired level of detail (microlevel), and development approach (iterative, focused on decreasing uncertainty from the top-down). Alternative modeling approaches that might be employed include Systems Dynamics (SD) models (but these do not allow for micro-level analysis) and Agent-Based Models (ABM) (but these are inherently more complex, because of the large number of variables within an agent model). Although ABM is more granular than DES and SD, it requires a detailed understanding of the model component characteristics and interactions and demands far more development and testing effort which is not feasible within the current constrains of this task. The level of abstraction using DES is optimal in that it will allow for development and analysis of a reasonably granular model within the task constraints.

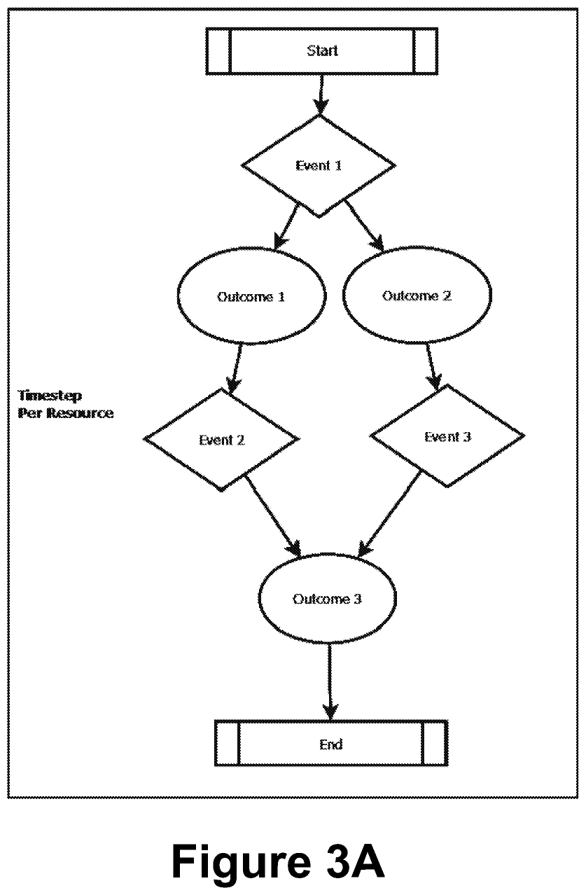

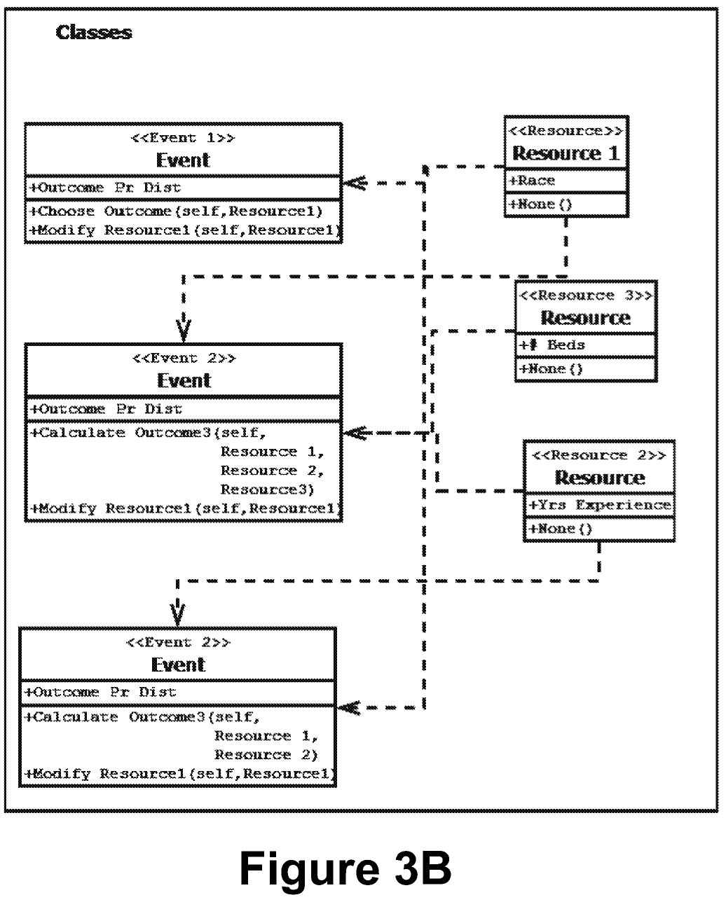

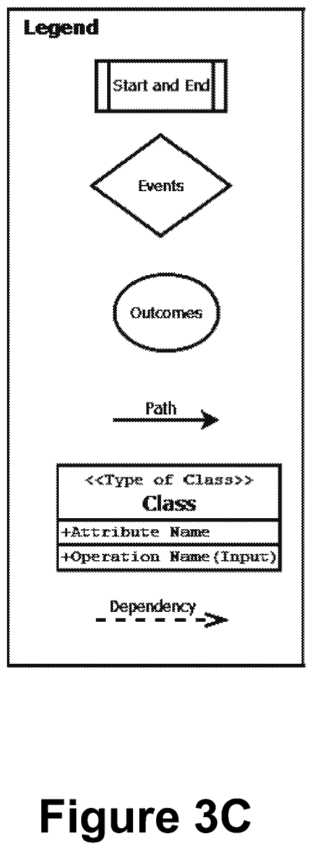

Some key definitions of the DES simulator are presented as follows. Table 5 presents some vocabulary used in describing the DES simulator. FIGS. 3A, 3B, and 3C present an example diagram showing elements of key simulator definitions and associated classes/attributes. FIG. 3A depicts a timestep of the simulation, including a timestep path running from “Start” to “Event 1” to “Outcome 2” to “Event 3” to “Outcome 3” to “End”. An alternative timestep path would run from “Start” to “Event 1” to “Outcome 1” to “Event 2” to “Outcome 3” to “End”. FIG. 3B diagrammatically shows illustrative resources having resource attributes and illustrative events having event attributes. FIG. 3C shows a legend of symbols used in the diagrams of FIGS. 3A and 3B.

TABLE 5
key vocabulary

| Name | Definition | Comments | Examples |
|---|---|---|---|
| Entities | Objects that have attributes, | Python Class | Patients, Doctors |
| (processes) | experience events, consume | | |
| | resources, and enter queues | | |
| | over time | | |
| Attributes | Features specific to an entity | Class attributes | Age, sex, race, |
| | that allow it to carry | | health status, |
| | information. | | past event |
| Events | Things that can happen to an | Initiated or | Clinical condition, |
| | entity or an environment. A | captured by class | adverse drug |
| | discrete element of the model | attributes, and | reaction, clinical |
| | with at least one outcome | methods, | decision, |
| | conditioned on a set of inputs. | simulation | treatment |
| | | Environment | |
| | | (SimPy Yield | |
| | | utilities) | |
| Resource | An object that provides a | Python Class | Building, |
| | service to an entity (may | | Organizations, |
| | require time). Attributes of | | etc. |
| | resource affect the probability | | |
| | distribution of an event | | |
| Queue | If a resource is occupied when | SimPy | |
| | entity needs it, then that entity | | |
| | must wait, forming a queue. | | |
| | Queues can have a maximum | | |
| | and alternative approaches to | | |
| | calling entities from queues, | | |
| | can be defined. | | |
| Time | An explicit simulation clock | SimPy | Hard to think of |
| | keeps track of time. | | examples, pretty |
| | Referencing this clock makes | | fundamental to |
| | it possible to track interim | | the universe. |
| | periods. (Discrete handling of | | |
| | time means that the model | | |
| | can efficiently advance to the | | |
| | next event time.) | | |
| Interaction | Entities competing over a | Specified with the | Patients waiting |
| | resource. | class attributes, | for a service |
| | | and methods, and | |
| | | simulation | |
| | | Environment | |
| | | (SimPy) | |
| Outcome | The result of an event, may be | | Patients |
| | qualitative (i.e., a yes/no | | diagnosed with |
| | decision) or quantitative (i.e., | | flu, Time waiting |
| | modeling the time to recovery | | for doctor |
| | for an illness once all care | | |
| | decisions are completed.) | | |
| Path | The connection between two | | |
| | events. Nothing happens on a | | |
| | path, but it serves to connect | | |
| | events. | | |
| Timestep | A period of time over which a | | Episode of care |
| | set of events are expected to | | |
| | occur in a defined order. | | |
| | Incorporates elements of time, | | |
| | events, and paths. | | |
| Timestep | All of the events experienced | | During my |
| Path | by a given entity in a timestep. | | episode of care . . . |
| Synthetic | An estimated population | | Population of |
| Population | constructed from known | | FQHC patients, |
| | features of a real population | | based on |
| | for the purposes of modeling | | empirical |
| | population dynamics. | | distributions |
| Scenario | A specific set of starting | | Assuming that x |
| | conditions and assumptions | | is set to 10 and y |
| | reflecting the state of the | | is set to 5 . . . |
| | simulator parameterization. | | |

There are two different approaches to developing the DES simulator. The first approach is to use off-the-shelf DES simulation software, and the alternative is to develop the simulation software using an open source software platform such as Python. The first option allows for developing a prototype model very quickly, with minimal software development work required, allowing more time for testing and model refinement. The latter approach allows for developing a more flexible and configurable simulation tool that can be modified more readily to solve problems that are too unique or specialized to be modeled using an off-the-shelf software tool easily. The approach employed in the illustrative embodiments is to build the tool in Python, after performing a short performance test and deciding that flexibility of data inputs and outputs in Python. In both cases, it is possible to specify a set of features desired in a simulation tool. The overall goal is to support rapid prototyping (i.e. limited compile times, simple interface, integrated documentation, etc.), research reproducibility (plain text, version controlled inputs, outputs, and code), and speed. The simulator is responsible for simulation runs (not data preparation or data analysis) and exporting data into a format suitable for archiving and entering into version control to support reproducibility. Accordingly, the simulator preferably utilizes software that: is object-oriented; modular, allowing for objects to be added and used selectively depending on modeling scenario; extensible, allowing new functionality to be added as needed; has utilities for unit testing, including a coverage checker (to assess the proportion of code that has associated tests); has utilities for system testing, allowing for discrete test cases demonstrating end-to-end functionality of the simulator; is platform independent (i.e. compiles and runs on any environment), or can be made platform independent (i.e. can be compiled, then deployed to run), specifically between a Linux and Windows environment; can run in parallel, allowing multiple scenario runs in parallel (for uncertainty testing); has utilities for data visualization; is suitable for quick prototyping; is free or open source; has developer tools designed to ease development, build dashboards, and perform other maintenance tasks; has an existing, broad library of open source libraries to ease development; and provides exception handling and a simple logging infrastructure.

One existing software tool is called AnyLogic which is a multimethod simulation tool supporting DES, ABM and SD simulation approaches. This software comes with a graphical user interface (GUI) modeling language and is extensible using Java code. The code software contains built in libraries, including the Process Modeling Library, the Pedestrian Library, the Rail Library, and the Fluid Library. It also supports 2D and 3D animation. The model can be exported as a Java application that be run separately or integrated with other software. Battelle currently has an active single license on a common computer (e.g. PC) accessible by team members for this project. The primary requirements to use this tool to develop a simulation tool is run it on a single common PC, which a Windows system.

In the following, some quality and cost/benefit considerations are discussed, as regards certain practical considerations in selecting a platform for implementing the DES. A suitable open-source toolset for this task is Python which can run on one or more computers having a Windows, Linux, and/or MacOS operating system. By way of illustration the Linux platform is assumed, e.g. with style checking by Pylint configured to match a chosen Style Guide defined in Python Style Guides and Best Practices. The unit testing and code coverage checking will be an integral part of the development phase using Python's unittest testing framework. By way of nonlimiting illustrative example, the Anaconda python distribution is suitable, with Python version 3.6. The choice of integrated development environment (IDE) suitably depends on personal preferences if the other requirements are met. Some options are PyCharm, and PyDev. Version control will be handled with Git. A ‘semi-agile’ development method for task management as defined in the Assumptions: Development Approach. Table 6 provides a comparison of AnyLogic and Python software based on specified characteristics.

TABLE 6
Comparison of AnyLogic and Python software

| Requirement | AnyLogic | Python |
|---|---|---|
| Object-oriented | Yes, with pre-specified | Yes, no pre-specified |
| | object types and ability | object types |
| | to build new ones; Java | |
| Modular | Yes | Yes |
| Extensible | Yes (using Java | Yes |
| | integration) | |
| Unit testing | Unknown | Yes, unit-testing library. |
| | | Coverage checks with |
| | | “Coverage” app |
| System testing | Unknown | Yes, same as unit testing |
| Platform | Yes, using native Java | Yes; Cannot be compiled |
| independence | implementation; Can | and run as standalone |
| | compile models and | application |
| | run as a standalone | |
| | application; As | |
| | currently deployed, | |
| | only on Windows | |
| Can run in | Yes; As currently | Yes, with limitations |
| parallel | deployed, no | imposed by Global |
| | | interpreter Lock, other |
| | | C-implementation |
| | | libraries or a specific |
| | | job-dispatcher |
| Utilities | Yes, fully integrated | Libraries to support |
| for data | | building data |
| visualization | | |
| Suitability | Yes, intuitive user | Yes, simple integrated |
| for quick | interface, intended for | documentation, no |
| prototyping | rapid prototyping | graphical user interface |
| Free or | No. Quite expensive with | Yes |
| open-source | restrictive licensing rules | |
| Developer | Intuitive GUI for model | Great tools, no GUI |
| Tools | development and to | |
| | support analysis | |
| Existing, broad | Yes, but undetermined | Yes, but mostly at a |
| library of open | appropriateness | basic level requiring |
| source libraries | | development |
| Provides exception | Unknown | Yes, a key design |
| handling and | | component of Python |
| simple logging | | |

The primary trade-off between AnyLogic and Python relates to the availability of the Graphical User Interface (GUI) vs. an open-source approach. One comes with a real cost in terms of purchase price and limitations of portability (AnyLogic), and the other comes with cost related to staff development time. Over a long period, the in-house Python solution is likely to result in a new, innovative approach with great flexibility (while incurring maintenance and new development costs), while AnyLogic is likely to incur lower costs in terms of maintenance and new development, but higher actual costs for ownership. In the short term, the Python solution will be costlier to develop new models, but will gain economies of scale and a unique capability. In the short term, AnyLogic imposes constraints (although limited) on model design, but gains in economy of scale will be smaller (because many of those improvements to the libraries have already been made).

Another task in the development process is to cross check and test the simulation results with the ‘truth’ values. To that end, a set of specific use cases are suitably developed, some of which address edge cases and specific scenarios that can be used to check the veracity of the simulation results. The ‘truth’ values for the use cases may be developed using R or excel spread sheets.

Data requirements are next described. The illustrative modeling domain is primarily claims data, as it is commonly used for reporting pilot effectiveness and will map well to reports of impact from pilot evaluations (for reproducibility), the information processing needs of our potential clients, and available empirical data. Additionally, survey data may be utilized when necessary (if no claims-based outcome measures are available) and population-level data as needed (e.g. census, population risk factors, etc.).

The data needed to parameterize the models differs from the data needed to replicate real-world evaluation findings. In each case, it is not necessary to have raw data containing identifiable information. Instead, synthetic datasets drawn from the real-world probability distributions (but simulated) or information about the distribution of a given dataset or set of outcomes can be utilized.

Data for parameterization is suitably defined at the event level. For each event, there are several risk factors and a pre-defined probability distribution. If we treat each event initially as having a uniform probability across outcomes, that maximizes uncertainty. Any data analysis that can affect the assumption of uniform probability should serve to reduce uncertainty. Because each event is considered independent, it falls to the model developer to select the most appropriate dataset to build the probability distribution. Where data is not available, and information exists to improve upon the uniform probability distribution, then that information will be utilized. Potential sources of information include (in prioritized order): (1) Peer-reviewed summary data or coefficients with estimates of uncertainty (e.g. standard errors); (2) internal analysis of datasets (possibly replicating findings from sources that are not peer-reviewed); (3) Estimates of probability from a large, independent crowd of experts (using well-established estimation methods, such as Delphi, and maintaining extensive documentation); and (4) Estimates from a small group, or an individual expert (using well-established estimation methods, such as Delphi, and maintaining extensive documentation). The process for incorporating data for parameterization may suitably include at least one within-team peer review to validate assumptions, quality, and level of documentation.

Data for replication may be based on the specific outcomes identified in evaluation reports from pilot evaluators. These outcomes are preferably reported quantitatively, and allow for direct comparison with the outcomes modeled by the HTIL RPM. Outcomes that are qualitative may still be desirable if they are the only tool available.

Supporting infrastructure in the case of AnyLogic includes Common PC access and a software license. Supporting infrastructure in the case of python includes: Version Control (e.g. Git server); Machines for model runs; and Machine for cross checking and development work, in addition to HPC, include Oracle VM Virtual Box and Linux Virtual Machines and personal computers (used for R). Both Python and R are open source packages and are readily available for downloading. Furthermore, the rapid prototyping system software and associated parameters and other DES data are suitably stored on a non-transitory storage medium that is readable and executable by the computer to implement the DES simulator and associated outcomes analysis (e.g. aggregating outcomes for the population or generating disaggregated outcomes for defined segments of the population, performing uncertainty analysis, and so forth). The non-transitory storage medium may, for example, comprise a hard disk or other magnetic storage medium, a solid state drive (SSD) or other electronic storage medium, an optical disk or other optical storage medium, various combinations thereof, or so forth.

Table 7 presents some illustrative user stories for development, descriptions, priority and status.

TABLE 7
User Stories

| Key | Summary |
|---|---|
| HTIL-597 | change numberOfAnnualTrainings from constant to uniform |
| | draw and include it in the json output file. |
| HTIL-595 | updateRunTimeParameters.csv in rpm-scenarios repo |
| HTIL-593 | Modify the implementation of the doctor capability multiplier |
| HTIL-585 | As a user. I can “delete” my own scenarios or experiments, |
| | but they are only hidden from view |
| HTIL-576 | As a user. I can click on a parameter in the scenario builder |
| | and see the distribution and |
| HTIL-575 | As a user, I can download an HPC batch file for an |
| | experiment |
| HTIL-574 | As a user, I can download a zipped file of the experiment |
| | design |
| HTIL-573 | As a user, I can launch an experiment from the experiments |
| | view page |
| HTIL-572 | As a user, I can browse public and my private experiments |
| | in a table view (experiments view) |
| HTIL-571 | As a user, I can create an “experiment” from a set of |
| | population and runtime scenarios |
| HTIL-570 | As a user, I can create runtime scenario based on the current |
| | version of RPM |
| HTIL-569 | As a user, I can create a population scenario based on the |
| | current version of RPM |
| HTIL-567 | As a user, I can login to a web interface |
| HTIL-447 | Parameterize Wellness Check Initial Conditions |
| HTIL-314 | Plot of no show, go to ED, stay home vs. day |
| HTIL-313 | Scatter plot of appointment time per day |
| HTIL-312 | Plot number of appointments per day |
| HTIL-149 | As a user, I should be able to specify a set of scenarios to |
| | run a qualitative uncertainty characterization |
| HTIL-148 | As a user, I should be able to specify and run uncertainty |
| | characterization using a monte-carlo approach |
| HTIL-147 | As a developer, I should be able to iterate though a series |
| | of events, defined as a single Timestep path where state |
| | information is passed through synthetic |
| HTIL-146 | As a developer, I need a structured way of generation event |
| | modules |
| HTIL-145 | As a user, I should be able to get data out of the simulation |
| | in a structured, ready to analyze format |
| HTIL-144 | As a developer, I can specify ranges with specific likelihoods |
| | for all inputs and outputs on events |
| HTIL-143 | As a developer, I can specify an arbitrary number of events, |
| | with linkages to other paths for a simulation model run |
| HTIL-142 | As a user, I can pass a synthetic population to the simulator |
| | and have a graceful failure |
| HTIL-141 | As a developer, I can build a synthetic population from a set |
| | of known distributions |
| HTIL-140 | As a user, I should be able to input starting data from a |
| | pre-computed scenario |

For the purposes of replication, HTIL RPM may be judged against the reported metrics from the relevant pilot evaluation report. A scoring matrix consisting of a 0-1 score for each dimension may be utilized. The accuracy measure will be calculated by dividing the total score from the scoring matrix, by its maximum score. Over time, the objective of the modeling approach will be to improve this score. For the purposes of replication analysis, we are interested in how the scores were generated and better understanding differences. The following heuristics will be used for scoring: Qualitative outcomes (i.e. binary) will be scored using log loss, e.g. normalized onto a 0-1 scale, where 1 is better; and Quantitative results (i.e. continuous or count). The quantitative results may be compared qualitatively by assessing whether the simulated results and the actual results from the pilot evaluation overlaps using 95% confidence intervals.

While described with respect to the U.S. healthcare policy development and healthcare cost management domains, it will be appreciated that the disclosed DES simulators may find application in a wide range of other business domains, such as transportation policy development, transportation system cost management, educational policy development, educational system cost management, and so forth.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A rapid prototyping system for prototyping a process in a business domain, the rapid prototyping model comprising:
a computer programmed to perform discrete event simulation (DES) of a scenario in the business domain, the DES being defined by a plurality of resources having resource attributes, a plurality of events having event attributes, and a plurality of entities having entity attributes, the computer programmed to implement a DES simulator configured to run the scenario including performing time steps per resource in which the entities experience events.

2. The rapid prototyping system of claim 1 wherein the computer is further programmed to generate a synthetic population of entities upon which the DES simulator runs the scenario.

3. The rapid prototyping system of claim 2 wherein the computer is further programmed to generate outcomes for the scenario based on the running of the scenario.

4. The rapid prototyping system of claim 3 wherein the computer is programmed to run:
a baseline scenario that does not include a proposed innovation to the business domain, and
an innovation scenario that includes the proposed innovation to the business domain;
wherein the generated outcomes include at least one comparison of the outcomes for the innovation scenario versus the outcomes for the baseline scenario.

5. The rapid prototyping system of claim 4 wherein the computer is further programmed to perform uncertainty analysis on the generated outcomes for the scenario.

6. The rapid prototyping system of claim 5 wherein the uncertainty analysis comprises a scenario-based uncertainty analysis in which predefined scenarios are run by the DES simulator to test limits of the innovation.

7. The rapid prototyping system of claim 5 wherein the uncertainty analysis comprises a Monte Carlo-based uncertainty analysis in which scenarios generated by Monte Carlo sampling are run by the DES simulator to generate distributions of the outcomes.

8. The rapid prototyping system of claim 3 wherein the outcomes for the scenario include aggregated outcomes for the population and disaggregated outcomes for defined segments of the population.

9. The rapid prototyping system of claim 3 wherein the computer includes a display on which the generated outcomes are presented.

10. The rapid prototyping system of claim 1 wherein the DES simulator is implemented in Python.

11. The rapid prototyping system of claim 1 wherein the business domain is a healthcare domain.

12. The rapid prototyping system of claim 1 wherein the business domain is the United States healthcare domain.

13. A rapid prototyping method for prototyping a process in a business domain, the rapid prototyping method comprising:
using a computer, running a discrete event simulation (DES) of a scenario in the business domain, the DES being defined by a plurality of resources having resource attributes, a plurality of events having event attributes, and a plurality of entities having entity attributes, the running of the DES of the scenario including performing time steps per resource in which the entities experience events; and
generating outcomes for the scenario based on the DES of the scenario.

14. The rapid prototyping method of claim 13 further including:
using the computer, generating a synthetic population of entities upon which the computer runs the DES of the scenario.

15. The rapid prototyping method of claim 13 wherein the computer is programmed to run DES of:
a baseline scenario that does not include a proposed innovation to the business domain, and
an innovation scenario that includes the proposed innovation to the business domain;
wherein the generated outcomes include at least one comparison of the outcomes for the innovation scenario versus the outcomes for the baseline scenario.

16. The rapid prototyping method of claim 13 further comprising:
using the computer, performing uncertainty analysis on the generated outcomes for the scenario.

17. The rapid prototyping method of claim 16 wherein the uncertainty analysis comprises a scenario-based uncertainty analysis in which the computer runs DES of predefined scenarios to test limits of the innovation.

18. The rapid prototyping method of claim 16 wherein the uncertainty analysis comprises a Monte Carlo-based uncertainty analysis in which the computer runs DES of scenarios generated by Monte Carlo sampling to generate distributions of the outcomes.

19. The rapid prototyping method of claim 13 wherein the business domain is a healthcare domain.

20. A non-transitory storage medium storing instructions readable and executable by a computer to perform a rapid prototyping method comprising:
using a computer, running a discrete event simulation (DES) of a scenario in the business domain, the DES being defined by a plurality of resources having resource attributes, a plurality of events having event attributes, and a plurality of entities having entity attributes, the running of the DES of the scenario including performing time steps per resource in which the entities experience events; and
generating outcomes for the scenario based on the DES of the scenario.
